(19) United States
(12) Patent Application Publication
(10) Pub. No.: US 2023/0165911 A1
(43) Pub. Date: Jun. 1, 2023

(54) COMPOSITION AND METHOD FOR TREATING EQUINE SARCOIDS

(71) Applicant: Mary Hartman, Rochester, MN (US)

(72) Inventor: Mary Hartman, Rochester, MN (US)

(21) Appl. No.: 17/539,848

(22) Filed: Dec. 1, 2021

(51) Int. Cl. A61K 35/644; A61P 35/00; A61K 35/748; A61K 36/537

(52) U.S. Cl. CPC A61K 35/644; A61K 35/748; A61K 36/537; A61P 35/00

(57) ABSTRACT

A composition and method for treating equine sarcoids includes providing a composition ingestible by a horse wherein the composition comprises Manuka honey, chia seeds, and spirulina powder. A dosage, being an effective amount of the composition, is fed to a horse having one or more equine sarcoids.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### Field of the Invention.

The disclosure relates to treatments for equine sarcoids. Equine sarcoids are a type of skin tumor which comprise the most common skin tumor found in horses. Such tumors are not malignant, but they do spread quickly, cause irritation, can bleed easily if disturbed, and thereafter can become easily infected. Prior treatments have included, for example, surgical removal with a scalpel, binding the sarcoid with a band to prevent blood flow to the sarcoid, freezing, radioactive treatments, chemotherapy, and vaccinations injected into the tumors. Each of these methods is only somewhat effective in the removal or shrinkage of the tumors and many include undesirable side effects including possible death such as from anaphylactic shock from the vaccines. Thus, more effective treatments and preventatives are needed which also provide for lessening of treatment side effects.

### Description of Related Art Including Information Disclosed Under 37 CFR 1.97 And 1.98.

The prior art relates to conventional treatments utilized for removing or retarding the growth of equine sarcoids.

### BRIEF SUMMARY OF THE INVENTION

An embodiment of the disclosure meets the needs presented above by generally providing a composition ingestible by a horse wherein the composition comprises Manuka honey, chia seeds, and spirulina powder. A dosage, being an effective amount of the composition, is fed to a horse having one or more equine sarcoids.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWINGS

Not Applicable

### DETAILED DESCRIPTION OF THE INVENTION

With reference now to the below, a new equine sarcoid treatment embodying the principles and concepts of an embodiment of the disclosure will be described.

The composition and method for treating equine sarcoids generally comprises a composition which will be ingested by a horse, at an effective dosage, over an amount of time required as may be affected by the type of sarcoid, the extent to which the sarcoid has invaded the body, and the response by of the horse being treated. The final composition fed to a horse may contain any edible supplements having various nutritional value but will at least include Manuka honey, chia seeds, and spirulina powder, mixed together and provided to a horse for ingestion.

Manuka honey is ubiquitous and contains at least a portion of nectar from the Manuka tree, Leptospermum scoparium. More particularly, the Manuka honey will have a UMF rating of at least 15+. UMF is standard for “Unique Manuka Factor” and is a measurement of three signature compounds found in Manuka honey. The three compounds are methylglyoxal, dihydroxyacetone, and leptosperin. In some Manuka honey validation test, hydroxymethylfurfural may also be measured to test for age of the honey and that the temperature of the honey has not been manipulated. The measurement is a standard measurement used by the Unique Manuka Factor Honey Association and typically provides a measurement of 0 to 25+ though higher ratings are achievable. Other rating systems are available and include the MGO scale (methylglyoxal level) and the NPA scale (non-peroxide activity). Both MGO and NPA levels correspond generally to the UMF ratings and a UMF rating of 15+ is approximately at least 514+ on the MGO scale and 15+ on the NPA scale.

Chia seeds, which comprise seeds from the Salvia columbariae or Salvia hispanica plants, are typically provided directly as seeds or may be ground into a flour or paste. Spirulina powder is a biomass of cyanobacteria including Anthrospira platensis.

In one embodiment the composition includes by weight:
from 1 part to 20 parts Manuka honey;
from 5 parts to 15 parts chia seeds; and
from 4 parts to 30 parts spirulina powder.

In another embodiment the composition includes by weight:
from 1 part to 2 parts Manuka honey;
from 8 parts to 12 parts chia seeds; and
from 4 parts to 12 parts spirulina powder.

It should be understood that other fillers may be added to the above compositions for the purpose of adding nutritional value including vitamins and minerals, as well as binding agents depending on desired form the composition will be provided. Thus, the composition may be part of feed or may be provided in caplets, pressed cakes, and the like.

The composition is provided in a dosage that is provided to the horse at least once daily over an extended period of time. The extended period of time will generally be 60 days or greater. The dosage will include from 15 grams to 100 grams of the composition, exclusive of any additional fillers. That is, the combined weight of the Manuka honey, chia seeds, and spirulina powder will be from 15 grams to 100 grams. Since the composition comprises ingredients that are all readily and safely digestible by a horse, there is no danger in providing greater amounts of the composition and the 15 grams to 100 grams of composition per dosage is reflective of an effective amount of the composition for treating equine sarcoids. The composition, once formulated, is fed to a horse suffering from one or more equine sarcoids.

### Test Results

A test study was performed utilizing a Chia/Spirulina/Manuka composition for the Treatment of Equine Sarcoids.

### Materials and Methods

Three horses with a total of 10 sarcoids were enrolled in the trial. Two horses were mares, and one was a stallion. The mares were 6 and 8 years old respectively and the stallion was 15 years old. The 6 year old mare presented six sarcoids, the 8 year old mare two sarcoids, and the stallion two sarcoids. Additional to note is that the sarcoids on the stallion had been previously treated with a medicated cream of unknown components, while the combined 8 sarcoids on the mares had never received medical treatment. There had been no prior surgical interventions on any of the sarcoids and all sarcoids had been present from 4 to 5 years.

Prior to the initiation of the treatment trial, all presented sarcoids were measured by a licensed veterinarian. Measurements were made along the longest axis of the lesion and along the orthogonal place of the long axis. These two measurements were used to calculate the cross-sectional area of the lesion. The depth of the lesions was not recorded as the subcutaneous extent of sarcoids cannot easily be determined visually. The horses were each fed between 55 grams and 60 grams of the composition daily for four months. The specific composition for the test trial consisted of 10.3 grams of chia seeds, 1.2 grams of +16 Manuka honey, and 5.0 grams of spirulina powder. The horses otherwise continued their standard feed regimen with no additional sarcoid treatments. After the termination of the four-month trial, the sarcoid lesions were measured again along the same axis as the initial measurements, and the cross-sectional area was again calculated. The percentage change in size of each lesion was then calculated using the equation (A2-A1)/A1x100.

Results

| 8 Year Old Mare | | | |
|---|---|---|---|
| Sarcoid Location | Beginning Size | End Size | % Change |
| Left Leg | 3130 mm^2 | 3229.9 mm^2 | 3.2 Increase |
| Right Leg | 141 mm^2 | 0 | Resolved |
| 6 Year Old Mare | | | |
| Sarcoid Location | Beginning Size | End Size | % Change |
| Left Eye | 601.7 mm^2 | 161.7 mm^2 | 73.1 Decrease |
| Left Zygomatic Arch | 514.1 mm^2 | 255.2 mm^2 | 50.4 Decrease |
| Left Chin | 805.2 mm^2 | 285.7 mm^2 | 64.50 Decrease |
| Right Side of Arch | 81.5 mm^2 | 0 | Resolved |
| Right Chest | 770.3 mm^2 | 74.8 mm^2 | 90.3 Decrease |
| Left Flank | 1372 mm^2 | 478.1 mm^2 | 64.5 Decrease |
| 15 Year Old Stallion | | | |
| Sarcoid Location | Beginning Size | End Size | % Change |
| Left Rib Cage | 1842.1 mm^2 | 803.2 mm^2 | 56.5 Decrease |
| Left Flank | 3526.4 mm^2 | 3085.8 mm^2 | 13.4 Decrease |

### DISCUSSION OF RESULTS

After 4 months of treatment, 9 of the 10 sarcoid lesions had decreased in size and 2 of the 9 resolved completely. Only one sarcoid increased in size. The nine lesions that had decreased in size had an average decrease in cross-sectional area of 68.1%. The one lesion that had increased in size had an increase in cross-sectional area of 3.2%. The overall percentage change in cross-sectional area of all 10 lesions was a decrease of 61%.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A method of treating equine sarcoids, said method including the steps of:
providing composition ingestible by a horse, said composition comprising:
Manuka honey;
chia seeds; and
spirulina powder;
feeding a dosage of the composition to a horse having one or more equine sarcoids.

2. The method of treating equine sarcoids as in claim 1, wherein the Manuka honey has a UMF rating of at least 15+.

3. The method of treating equine sarcoid as in claim 1, wherein the composition includes a ratio of:
from 1 part to 20 parts Manuka honey;
from 5 parts to 15 parts chia seeds; and
from 4 parts to 30 parts spirulina powder.

4. The method of treating equine sarcoid as in claim 1, wherein the dosage comprises from 15 grams to 100 grams of the composition.

5. The method of treating equine sarcoid as in claim 4, wherein the step of feeding the dosage further includes feeding the dosage to the horse once a day for at least 60 days.

6. The method of treating equine sarcoid as in claim 5, wherein the composition includes a ratio of:
from 1 part to 2 parts Manuka honey;
from 8 parts to 12 parts chia seeds; and
from 4 parts to 12 parts spirulina powder.

7. The method of treating equine sarcoid as in claim 1, wherein the composition includes a ratio of:
from 1 part to 2 parts Manuka honey;
from 8 parts to 12 parts chia seeds; and
from 4 parts to 12 parts spirulina powder.
